SEQUENCE LISTING (9 sequences)

SEQ ID NO: 1 - DNA, 90 bp, Artificial Sequence
  CHAMP probe
gtttcccgttctaacggagttttggagggcgaacgcggctttccactagctaggccctacatgtctttcttccagtcgaggatgtttaca

SEQ ID NO: 2 - DNA, 24 bp, Artificial Sequence
  F-Chip primer
ccactagctatgtgacatgtaggg

SEQ ID NO: 3 - RNA, 22 nt, Homo sapiens
uguaaacauccucgacuggaag

SEQ ID NO: 4 - DNA, 78 bp, Artificial Sequence
  F-Chip primer extended
ccactagctatgtgacatgtagggcctagctagtggaaagccgcgttcgccctccaaaactccgttagaacgggaaac

SEQ ID NO: 5 - DNA, 20 bp, Artificial Sequence
  R-Chop primer
gtttcccgttctaacggagt

SEQ ID NO: 6 - DNA, 68 bp, Artificial Sequence
  B-chip primer, extended
gccgcgttctgtaggagggcgaacgcggctttccactagctaggccctacatgtcacatagctagtgg

SEQ ID NO: 7 - DNA, 20 bp, Artificial Sequence
  B-Chip primer
gccgcgttctgtaggagggc

SEQ ID NO: 8 - RNA, 22 nt, Homo sapiens
uguaaacauccuacacucagcu

SEQ ID NO: 9 - RNA, 22 nt, Homo sapiens
ugagguaguagguuguaugguu

(19) United States
(12) Patent Application Publication
(10) Pub. No.: US 2017/0335382 A1
(43) Pub. Date: Nov. 23, 2017

(54) Isothermal Amplification Assay for the Detection of Short Nucleic Acid Sequences

(71) Applicant: University of Pittsburgh - Of the Commonwealth System of Higher Education, Pittsburgh, PA (US)

(72) Inventors: Andrew John Bodnar, Pittsburgh, PA (US); Kathrin Gassei, Pittsburgh, PA (US); Jacqueline Ho, Pittsburgh, PA (US); Chandramohan Ishwad, Pittsburgh, PA (US); Abhay Narain Vats, Wexford, PA (US)

(21) Appl. No.: 15/525,458

(22) PCT Filed: Nov. 10, 2015
(86) PCT No.: PCT/US2015/059862

§ 371 Date: May 9, 2017

Related U.S. Application Data
(60) Provisional application No. 62/077,573, filed on Nov. 10, 2014.

(51) Int. Cl. C12Q 1/68

(52) U.S. Cl. CPC C12Q 1/6853

(57) ABSTRACT

Provided herein is a method of detecting short nucleic acids, such as microRNAs, in a sample, such as urine, and a kit for use in detection of the short nucleic acids.

### CROSS REFERENCE TO RELATED APPLICATIONS

This application claims the benefit of U.S. Provisional Patent Application No. 62/077,573, filed Nov. 10, 2014, which is incorporated herein by reference in its entirety.

### STATEMENT REGARDING FEDERAL FUNDING

This invention was made with government support under Grant No. AI082614 awarded by the National Institutes of Health. The government has certain rights in the invention.

Provided herein is a method of rapid detection of short nucleic acid sequences, such as microRNA (miRNA) sequences. The method offers a rapid, highly sensitive, specific and cost efficient alternative to current miRNA detection methods like qPCR, microarray, RNA sequencing, or in situ hybridization. The method also allows for the detection of target nucleic acid sequence in unprocessed biological samples, such as urine. A kit useful in implementing the method also is provided.

miRNAs are a class of single-stranded, non-coding RNA molecules, ranging in size from 20-30 nucleotides (nts), that regulate a variety of physiological functions. They play critical roles in physiological processes ranging from cell proliferation, differentiation, apoptosis, to tumor metastasis, as well as others. Circulating miRNAs are highly stable and are both detectable and quantifiable in a range of accessible biologic fluids, making them excellent candidates for diagnostic and prognostic biomarkers, for a variety of diseases.

An accurate analysis of miRNA expression is desirable not only for their role as biomarkers but also for the understanding of miRNA function, especially since miRNAs fine-tune the expression of most of their target miRNAs. However, given the fact that mature miRNAs are short in length and some miRNAs contain highly homologous sequences, the characterization of miRNA expression patterns remains a challenging task. A great deal of effort has been made in developing miRNA detection methods, including modified Northern blot, invader assay, rolling circle assay (RCA), splinted ligation, and locked nucleic acid (LNA) probe-based assays, in addition to real-time RT-PCR-based approaches. Most of these miRNA detection assays involve multiple steps, or are technically challenging, expensive, and time consuming. For example, RCA involves multiple steps including: reverse transcription of miRNA, circularization, C-Probe ligation, and amplification. RT-PCR of miRNA is based on expensive probe chemistry and multiple steps including reverse transcription and requires specialized equipment. Additionally, histological analysis of miRNA signatures require in situ hybridization which currently is very time consuming and can be error prone.

### SUMMARY

Provided herein is fast and reliable assay and kit for nucleic acid, e.g., microRNA, detection and amplification that is inexpensive, requires minimal to no sample preparation, and also requires minimal instrumentation.

According to one aspect of the invention, a method is provided for of identifying the presence of and/or the amount of a target nucleic acid in a sample. The method comprises: a) adding a sample, such as a biological sample, to a reaction mixture comprising a thermostable DNA polymerase having strand displacement activity and no 5′-3′ exonuclease activity; deoxyribonucleotide triphosphates; a nucleic acid or nucleic acid analog probe comprising a 3′ portion and a 5′ portion, wherein the 3′ portion is a sequence that binds to or is fully complementary to the target nucleic acid, and the 5′ portion comprises in a 3′ to 5′ direction a first primer binding site, a second primer binding site spaced apart from the first binding site, and a third primer binding site; a first primer having a 3′ portion and a 5′ portion and a first tag at its 5′ end, where the 3′ portion of the first primer binds to or is fully complementary to the first primer binding site and the 5′ portion does not bind to the probe; a second primer having a 3′ portion and a 5′ portion and a second tag, the same as or different from the first tag, at its 5′ end, where the 3′ portion of the second primer has the sequence of the second binding site or binds to a sequence fully complementary to the second binding site and the 5′ portion does not bind to a sequence fully complementary to the probe; and a third primer either having the sequence of the third binding site or binding a sequence complementary to the third binding site, and a third tag at its 5′ end that is different from the first and second tags; b) incubating the reaction mixture at a temperature effective to produce reaction product from the polymerase in the presence of the target nucleic acid; and c) determining the presence of, and optionally quantifying production of reaction product in the sample by detecting and/or quantifying nucleic acids larger than the first and second primer tagged with the first and second tags. In one aspect, the probe further comprises a) one or both of a fourth and fifth primer binding site, that optionally are between the first and second binding sites on the probe, and optionally, the fourth primer binding site also is 3′ to the fifth primer binding site; and b) the 5′ portion of the first primer has the sequence of the fourth primer binding site or binds to a sequence fully complementary to the fourth binding site and/or the 5′ portion of the second primer binds to or is fully complementary to the fifth binding site. In another aspect, the target nucleic acid is an RNA, and the thermostable DNA polymerase having strand displacement activity and no 5′-3′ exonuclease activity thermostable DNA/RNA polymerase without exonuclease activity can elongate from an RNA primer. In another aspect, the 5′ portion of the probe does not bind a nucleic acid of the biological sample and/or of the organism from which the biological sample is obtained. In yet another aspect, the temperature effective to produce reaction product is from 60° C. to 70° C. In one aspect of the method the sample is a biological sample, such as a bodily fluid, such as urine or blood, or purified nucleic acid.

The product of the reaction may be detected and optionally quantified by any useful method. In one aspect, the target nucleic acid is detected by gel electrophoresis. In another, the reaction product is detected by an in situ method, for example where the sample is an organ, a tissue sample or a section of a tissue sample on a slide and the reaction mixture is applied to the sample and is incubated and detected in-situ. The sample is, in one aspect, obtained from a kidney. One or more steps of the described methods may be automated.

In one aspect, the 5′ portion or the probe has the sequence 5′-GTTTCCCGTT CTAACGGAGT TTTGGAGGGC GAACGCGGCT TTCCACTAGC TAGGCCCTAC ATGTCTTT 3′ (SEQ ID NO: 1, bases 1-68); the 3′ portion of the probe has the sequence 5′-CTTCCAGTCGAGGATGTTTACA-3′ (SEQ ID NO: 1, bases 69-90), or the probe has the sequence 5′-GTTTCCCGTT CTAACGGAGT TTTGGAGGGC GAACGCGGCT TTCCACTAGC TAGGCCCTAC ATGTCTTTCT TCCAGTCGAG GATGTTTACA-3′ (SEQ ID NO: 1). In another aspect, the first primer has the sequence: 5′ CCACTAGCTATGTGACATGTAGGG-3′ (SEQ ID NO: 2); the second primer has the sequence: 5′ GCCGCGTTCTGTAGGAGGGC 3′ (SEQ ID NO: 7); and/or the third primer has the sequence: 5′ GTTTCCCGTTCTAACGGAGT 3′ (SEQ ID NO: 5).

According to another aspect of the invention, a kit is provided, comprising one or more one vessels, or a cartridge comprising compartments, comprising any of the primers and probe described herein. In one aspect, the kit further comprises: a reaction mixture comprising: a thermostable DNA polymerase having strand displacement activity and no 5′-3′ exonuclease activity, and optionally being capable of elongating from an RNA primer; and deoxyribonucleotide triphosphates (e.g., a mixture of dATP, dTTP, dGTP, and dCTP). The kit, in one aspect also comprises a lateral flow device for detecting product of an isothermal reaction that proceeds in the presence of the target nucleic acid. In another aspect, the kit comprises a control sample of the target nucleic acid. In yet another aspect, the kit further comprises an agarose or acrylamide electrophoresis gel for use in detection of products of a reaction using the reaction mixture, for example, an agarose gel comprising ethidium bromide.

In yet another aspect of the invention, a method of determining the presence of nephrotic syndrome in a patient, is provided. The method comprises: a) obtaining a urine sample from the patient, b) incubating the urine sample in a reaction mixture comprising: a thermostable DNA polymerase having strand displacement activity, no 5′-3′ exonuclease activity, and which elongates from an RNA primer; deoxyribonucleotide triphosphates; a nucleic acid or nucleic acid analog probe comprising a 3′ portion and a 5′ portion, wherein the 3′ portion is a sequence that binds to or is fully complementary to a sequence 5′-UGUAAACAUCCUCGACUGGAAG-3′ (SEQ ID NO: 3), and the 5′ portion comprises in a 3′ to 5′ direction a first primer binding site, a second primer binding site spaced apart from the first binding site, and a third primer binding site; a first primer having a 3′ portion and a 5′ portion and a first tag at its 5′ end, where the 3′ portion of the first primer binds to or is fully complementary to the first primer binding site and the 5′ portion does not bind to the probe; a second primer having a 3′ portion and a 5′ portion and a second tag, the same as or different from the first tag, at its 5′ end, where the 3′ portion of the second primer has the sequence of the second binding site or binds to a sequence fully complementary to the second binding site and the 5′ portion does not bind to a sequence fully complementary to the probe; and a third primer either having the sequence of the third binding site or binding a sequence complementary to the third binding site, and a third tag at its 5′ end that is different from the first and second tags, c) incubating the reaction mixture at a temperature effective to produce reaction product from the polymerase in the presence of the target nucleic acid; and d) determining the presence of, and optionally quantifying production of reaction product in the sample by detecting and/or quantifying nucleic acids larger than the first and second primer tagged with the first and second tags. In one aspect, the probe has the sequence: 5′-GTTTCCCGTT CTAACGGAGT TTTGGAGGGC GAACGCGGCT TTCCACTAGC TAGGCCCTAC ATGTCTTTCT TCCAGTCGAG GATGTTTACA-3′ (SEQ ID NO: 1); the first primer has the sequence: 5′ CCACTAGCTATGTGACATGTAGGG-3′ (SEQ ID NO: 2); the second primer has the sequence: 5′ GCCGCGTTCTGTAGGAGGGC 3′ (SEQ ID NO: 7); and/or the third primer has the sequence: 5′ GTTTCCCGTTCTAACGGAGT 3′ (SEQ ID NO: 5).

### BRIEF DESCRIPTION OF THE DRAWINGS

FIGS. 1A and 1B are schematic illustrations of a generalized lateral flow device.

FIGS. 2A and 2B are two schematic diagrams showing primer binding site arrangement for two embodiments of the probe described below.

FIG. 3 is a schematic representation of the CHAMP miRNA assay (SEQ ID NOS: 1-6 as indicated in the Figure). For the sake of convenience, the strands are referred to as AS (Anti-sense) and S (sense, corresponding to the polarity of the target miRNA).

FIG. 4 is an illustration showing the probe design (SEQ ID NO: 1), specific for miR-30a-5p, in addition to the isothermal primers (F-Chip (SEQ ID NO: 2), B-Chip (SEQ ID NO: 7), and R-Chop (SEQ ID NO: 5)). The primers have a binding site for mature miR-30a-5p via a 20 nt sequence at the 3′-end, and were labeled with either biotin or digoxigenin on the 5′-end.

FIG. 5 is photograph of an agarose gel showing the temperature gradient used to determine the optimal CHAMP reaction temperature.

FIG. 6 is a graph showing the melting curves for miR-30a-5p, at a temperature of 64.5° C., 200 mM of MgSO4 concentration, and varying amounts of Bst polymerase.

FIG. 7 is a graph showing the melting curves for miR-30a-5p, at a temperature of 64.5° C., 200 mM of MgSO4 concentration, and varying amounts of Bst polymerase.

FIG. 8 is photograph of a 2% agarose gel showing that miR-30a-5p amplification is dependent on the Bst enzyme concentration.

FIG. 9 is a graph showing the melting curves for miR-30a-5p, at a temperature of 64.5° C., 16 U of Bst polymerase, and varying amounts of MgSO4.

FIG. 10 is a graph that shows the sensitivity of the CHAMP reaction on synthetic RNA dilution series. Reactions were done at a temperature of 64.5° C., 200 mM of MgSO4 concentration, and 16 U of Bst polymerase.

FIG. 11A shows that unprocessed urine spiked with synthetic miR-30a could be used in the reactions, without the need for RNA isolation. The presence of urine did not inhibit the reaction. FIG. 11B shows that the incorporation of biotin and digoxigenin in the primers allows for the detection of amplicons on lateral flow strips.

FIG. 12 are photomicrographs that show that CHAMP reactions can be used in-situ. Digoxigenin labeled F-Chip and B-Chip primers amplified targets and were visualized by incubating slides with an HRP-conjugated antibody. Samples were visualized by a color reaction development with BM purple.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases. “Comprising” and like terms are open-ended. The term “consisting essentially of” limits the scope of a claim to the specified materials or steps and those that do not materially affect the basic and novel characteristics of the claimed invention. The term “consisting of” excludes any element, step, or ingredient not specified in the claim. The terms “a” and “an” refer to one or more.

As described herein nucleic acids are biological macro-molecules, essential for all known forms of life. Nucleic acid molecules contain all of the necessary genetic material required by a given organism. Herein, reference to nucleic acids include both DNA (deoxyribonucleic acid) and RNA (ribonucleic acid), and are made from nucleotide monomers.

Nucleic acids have 3′ and 5′ ends. The 3′ end is the end terminating at the hydroxyl group of the third carbon in the sugar-ring. The 5′ end designates the end of the nucleic acid strand in which the fifth carbon in the sugar-ring of the deoxyribose or ribose is at the terminus, and often terminates in a phosphate group. A “nucleic acid analog” is a modified nucleic acid structure that can bind to a complementary sequence by Watson Crick base pairing, non-limiting examples of which include: phosphorothioate, peptide nucleic acids; locked nucleic acid (2′-O-4′-C-methylene bridge, including oxy, thio or amino versions thereof); unlocked nucleic acid (the C2′-C3′ bond is cleaved); 2′-O-methyl-substituted RNA; morpholino nucleic acid; threose nucleic acid; glycol nucleic acid; etc., as are broadly known. A locked nucleic acid (LNA), further refers to inaccessible RNA due to the modification of an RNA nucleotide. In one example, the ribose moiety of an LNA nucleotide is modified with an extra bridge connecting the 2′ oxygen and 4′ carbon, forming a bicyclic structure. The bridge “locks” the ribose in a 3′-endo conformation.

As used herein, “downstream to” or “3′ to” refers to a location in the direction of the 3′ end of the nucleic acid, while “upstream to” or “5′ to” refers to a location in the direction of the 5′ end of the nucleic acid.

Herein a “target nucleic acid sequence” is a nucleic acid sequence for which detection is being sought. Detection of a specific nucleic acid sequence may be useful in the diagnosis of a specific biological phenomenon, such as a particular disease. As indicated above, the target nucleic acid may be DNA, RNA (e.g., miRNA), or an analog thereof, such as a phosphorothioate, that can be elongated in the manner described herein with the described thermostable DNA polymerase.

Nucleic acids can be single-stranded or double-stranded. When double stranded, two single-stranded DNA strands bind, or hybridize, together, by Watson-Crick base pairing (e.g., A:T/U, T/U:A, G:C, or C:G). Two strands that bind or hybridize together are termed “complementary” and align in an antiparallel fashion meaning that the 5′ to 3′ orientation of each strand is opposite that of the complementary strand.

A sequence of nucleobases in a nucleic acid or nucleic acid analog that binds or hybridizes to another in any given reaction is sometimes not necessary 100% complementary (“fully complementary”, with perfect A:T, T:A, G:C and C:G matching through the entire strands), but can be less than 100% complementary, meaning that one or more nitrogenous bases (bases) of a strand do not align with a complementary base (e.g., mismatches, substitutions, deletions and insertions). In many amplification or polymerization reactions, there can be some mismatches present, yet the reaction can proceed. Thus a sequence of a primer or probe need not have 100% (full) complementarity to its binding sequence, though 100% is preferred in many instances. For purposes herein, unless indicated otherwise, where a nucleic acid is said to bind, or not bind another nucleic acid or sequence, it is meant that the binding or non-binding occurs in a reaction as described herein. “Binding” or “bind”, in reference to two nucleic acid strands refers to specific Watson-Crick base pairing.

Nucleic acid synthesis, as described herein, refers to the producing of an identical nucleic acid sequence from the original target nucleic acid and to producing a nucleic acid oligomer or polymer by synthetic methods. In one aspect, relating to the elongation of a nucleic acid primer bound to a template, it is the process by which copies of nucleic acid strands are generated from a target sequence. Synthesis of nucleic acids, in addition to the nucleic acid target sequence, requires nucleotide triphosphates (NTPs, e.g., dATP, dTTP, dGTP, dCTP, ATP, TTP, GTP, UTP, or other nucleotides, such as dITP), polymerase, MgSO₄, and at least one primer.

A “primer” is a single stranded nucleic acid oligomer that serves as a starting point for DNA/RNA synthesis by a DNA or RNA polymerase. The primer hinds to, or is fully complimentary to a specific DNA/RNA binding site, and binds in such a manner that its 3′ terminal base is bound to a target sequence, and a polymerase elongates the primer in a 5′ to 3′ direction using the complementary target sequence as a template. Primer binding sites are typically 5-15 bases in length, though they can be longer, as with the target nucleic acid binding site and the third primer binding site as described below. As indicated below, as seen with the described first and second primers, the primers can comprise additional sequences and tags, primarily or typically 5′ to the primer binding site, that do not bind to the same single-stranded nucleic acid that the primer binds.

Herein, the referenced “probe” is a nucleic acid or nucleic acid analog having a variable length, ranging, for example, from 75-2000 bases long, which is used in the detection of a nucleic acid target sequence. In the context of the methods, compositions and kits described herein, the probe has 3′ and 5′ portions, where the 3′ portion contains a nucleotide sequence that is complementary to a target nucleic acid target sequence to be detected such as an miRNA, and the 5′ portion is at least 70-80 bases and preferably has a sequence that does not bind to any nucleic acid of the sample to be tested. In another embodiment, especially where the sample comprises cells or genomic DNA, such as with an in situ assay, the 5′ portion of the probe also does not bind any nucleotide sequence from the genome of the organism from which the sample containing the target nucleic acid is taken.

A “tag” (alternately, “label”) is a detectable moiety attached, typically covalently, to a compound, which permits detection of the presence of the compound in any relevant assay. The tag is detectable, meaning that it has a measurable property or feature allowing for efficiency and ease in visualizing and/or quantifying specific products. Tags include: fluorochromes, dyes, radionuclides, enzymes, ligands, antigens, antibodies or other binding reagents, etc., as are broadly known. Non-limiting examples of suitable labels include biotin, digoxigenin (DIG), alkaline-phosphatase (AP) or horseradish peroxidase, as are used in the Examples below. In the context of the methods described herein, a “tag” in one aspect is attached covalently to a 5′ end of a specified nucleic acid primer molecule. In one aspect, a tag is a member of a binding pair; namely a moiety that binds specifically to another molecule, compound or composition, such as an antibody or other member of a binding pair, including, without limitation, biotin, digoxigenin, dinitrophenyl or a fluorescein, such as fluorescein isothiocyanate (FITC). In another aspect a tag is a moiety that is detectable without binding to another compound or composition as a binding pair. Examples of such tags include, without limitation: dyes or fluorophores (fluorochrome), such as FITC, cyanine dyes, rhodamine dyes, coumarin dyes, dyes; particles, such as gold, latex or carbon particles; quantum dots; or enzymes, such as alkaline-phosphatase (AP) or horseradish peroxidase (HRP). In certain aspects, a tag or handle facilitates isolation, immobilization, identification, or detection and/or increases solubility of a compound such as a nucleic acid or polypeptide. In various aspects, the tag may be a polypeptide, a polynucleotide (e.g., a nucleic acid ligand or aptamer), a carbohydrate, a polymer, or a chemical moiety, or any combinations or variant thereof. In certain embodiments, the tag is, for example, glutathione S-transferase (GST), protein A, protein G, calmodulin-binding peptide, thioredoxin, maltose binding protein, hemagglutinin (HA), myc, poly arginine, poly His, poly His-Asp or FLAG tags.

A “binding pair” is a pair of compounds or compositions (individually, “binding reagent”), such as, without limitation: a receptor or a portion or mimic thereof and its corresponding ligand; an antibody, antibody fragment, or antibody mimic and its corresponding antigen/epitope; an aptamer and its corresponding ligand; an affibody and its corresponding ligand; a nucleic acid or nucleic acid analog and a substantially or completely complementary nucleic acid or nucleic acid analog; or biotin and avidin/streptavidin, that bind to each other specifically. By specific binding, it is meant that in the context of an assay in which the binding takes place, such as in a lateral flow device, array, etc., the binding of one member of a binding pair to another member of the binding pair is exclusive at least to the degree that any non-specific binding does not interfere substantially with the particular assay.

In certain aspects, a binding reagent is an antibody or an antibody fragment. For example, an antibody may be monoclonal antibodies, or derivatives or analogs thereof, including without limitation: Fv fragments, single chain Fv (scFv) fragments, Fab′ fragments, F(ab′)2 fragments, single domain antibodies, camelized antibodies and antibody fragments, humanized antibodies and antibody fragments, and multivalent versions of the foregoing; multivalent activators including without limitation: monospecific or bispecific antibodies, such as disulfide stabilized Fv fragments, scFv tandems ((scFv)2 fragments), diabodies, tribodies or tetrabodies, which typically are covalently linked or otherwise stabilized (i.e., leucine zipper or helix stabilized) scFv fragments; receptor molecules which naturally interact with a desired target molecule.

In one aspect, the binding reagent is an antibody. Preparation of antibodies may be accomplished by any number of well-known methods for generating monoclonal antibodies. These methods typically include the step of immunization of animals, typically mice; with a desired immunogen (e.g., a desired target molecule-or fragment thereof). Once the mice have been immunized, and preferably boosted one or more times with the desired immunogen(s), monoclonal antibody-producing hybridomas may be prepared and screened according to well-known methods (see, for example, Owen, J. A. et al., KUBY IMMUNOLOGY, Seventh Edition, pp. 654-656, W.H. Freeman & Co. (2013), for a general overview of monoclonal antibody production). Production of antibodies and other binding reagents have become extremely robust. In vitro methods that combine antibody recognition and phage display techniques allow one to amplify and select antibodies or other binding reagents with very specific binding capabilities. These methods typically are much less cumbersome than preparation of hybridomas by traditional monoclonal antibody preparation methods. Binding epitopes may range in size from small organic compounds such as bromo uridine and phosphotyrosine to oligopeptides on the order of 7-9 amino acids in length.

In another aspect, the binding reagent is an antibody fragment. Selection and preparation of antibody fragments may be accomplished by any number of well-known methods. Phage display, bacterial display, yeast display, mRNA display and ribosomal display methodologies may be utilized to identify and clone desired antibody fragment activators that are specific for a desired target molecule, including, for example, Fab fragments, Fvs with an engineered intermolecular disulfide bond to stabilize the V_{H}-V_{L} pair, scFvs, or diabody fragments. Production of scFv antibody fragments using display methods, including phage, bacterial, yeast, ribosomal and mRNA display methods can be employed to produce the activator and/or selectivity component, as described herein. As described below, yeast display methods were used to produce an activator described below. Yeast display methods are described, for example, in Boder, et al. (2000) Proc. Natl. Acad. Sci USA 97:10701-5; Swers, et al. (2004) Nucl. Acids. Res. 32:e36; and Yeast Display scFv Antibody Library User's Manual, Pacific Northwest National Laboratory, Richland, Wash. 99352, Revision Date: MF031112.

Other examples of binding reagents that are well-known include engineered proteins, such as antibody mimetics, such as affibodies (modified protein A polypeptides), and nucleic acid ligands, also known as aptamers. Production of, identification of, and selection of engineered proteins and nucleic acid ligands are broadly-known, and are available commercially.

The term “high-throughput” refers to the use of multiplexing, specific robotics, fluidics, etc. and combinations thereof, to automate an assay as well as to process and screen many, for example tens, hundreds to thousands, of assays sequentially and/or concurrently. High-throughput screening allows a person of ordinary skill in the art to quickly conduct hundreds to millions of chemical, genetic, or pharmacological tests in as short a time possible with reduced risk of contamination and/or errors. High-throughput systems are meant to aid in the automation steps necessary for the methods discussed herein, not limited to but including transferring liquids, mixture of reaction materials, maintaining suitable environmental conditions, and obtaining, analyzing/converting, and displaying data. Non-limiting examples of high throughput systems include: PerkinElmer® FlexDrop™ IV, (PerkinElmer®, Shelton, Conn.) or the Lab Bot 800 Dual Arm Fully Automated Liquid Handling Workstation (FMP Laboratory Automation, Gilson Inc., Middleton, Wis.). Obtention, storage, analysis and output of data may be conducted by standard computer processes.

As used herein, a sequence that is said to bind to a specified sequence or region, such as a primer binding site of a nucleotide sequence, is a sequence that binds to that site under reaction conditions of the particular assay/reaction described, sufficiently to conduct the assay, and does not necessarily bind at all temperatures and under all conditions. The binding sequence is optionally fully complementary to the sequence of the binding site, recognizing that mismatches can be present and still result in binding sufficient to perform the stated reaction. Typically, there is at least 80% or at least 90% sequence identity between a primer and a binding site for the primer for the primer to function in a given reaction. The described binding sites are typically in the range of 5-15 bases in length. As would be readily understood by those of ordinary skill, the base content and length of any primer will dictate the binding or hybridization characteristics of the primer. Also, one of ordinary skill would readily understand that the methods presented herein are exemplified by the specific examples described below, but are generally applicable to most sequences that meet the described requirements, with each specific assay requiring optimization within the skill of an ordinary artisan in most occasions. The respective amounts of probe and each primer also can be determined by such optimization.

A “biological sample,” herein, refers to a sample obtained from an organism. Non-limiting examples of a biological sample comprise, blood, plasma, serum, cerebrospinal fluid (CSF), urine, lymph, ascites, saliva, semen, cell or tissue lysates or homogenates, etc.

As used herein, a “thermostable DNA polymerase capable of elongating from an RNA primer and having strand displacement activity and no 5′-3′ exonuclease activity” is understood to a person of ordinary skill in the art. The polymerase is thermostable, meaning it retains substantial activity at or above 60° C., and preferably over 70° C. By DNA polymerase, it is understood as an enzyme comprising the ability to add deoxyribonucleotide triphosphate (dNTP) residues to the 3′-end of the growing DNA chain, using a complementary DNA as template. Deoxyribonucleotide triphosphates (dNTPs) typically used for elongation are dATP, dTTP, dCTP and dGTP, but others may be utilized to optimize a particular reaction, such as deoxyinosine triphosphate.

The described DNA polymerase can elongate from a DNA or RNA primer bound to a DNA template and can displace, during polymerization, a nucleic acid that is bound to the DNA template 3′ to the polymerase. By 5′-3′ exonuclease activity, it is meant the ability of a polymerase, rather than displacing a bound nucleic acid, degrades the bound nucleic acid from the 5′ end. For Example DNA Polymerase I has a 5′-3′ exonuclease activity, but the Pol I “large fragment” therof does not. An example of a “thermostable DNA polymerase capable of elongating from an RNA primer and having strand displacement activity and no 5′-3′ exonuclease activity” includes, without limitation Bst DNA polymerase, large fragment, commercially available from New England Biolabs, Inc. of Ipswich, Mass. A second non-limiting example of a “thermostable DNA polymerase capable of elongating from an RNA primer and having strand displacement activity and no 5′-3′ exonuclease activity” is PyroPhage® 3173 DNA Polymerase, Wild Type, and Bst DNA Polymerase, Exonuclease Minus (also from Lucigen) commercially available from Lucigen Corporation of Middleton, Wis.

A “reaction mixture” is a mixture of stated ingredients and/or, at a minimum, a mixture of ingredients necessary for performing a reaction, such as the isothermal reaction described herein. Suitable reaction mixtures can include, for example, a buffer, salts, nucleotides, cofactors, primers, probes, target sequences and/or any other ingredient useful for performing an enzymatic reaction. In one example, such as that of the Example below, the reaction mixture, comprises: primers, probe, NTPs, polymerase, polymerase reaction buffer, betaine and MgSO₄.

“In situ hybridization” herein refers to a technique permitting the specific detection of nucleic acids molecules within individual cells, tissue sections or even whole mounts typically deposited on a solid support, in floating sections or an immersed cellular specimen.

A lateral flow device, as herein described, generally refers to device intended to detect the presence of, or absence of, a target analyte in a given biological sample. Examples of lateral flow devices are broadly known. For example, a lateral flow device can be described as an immunoclaromatographical test device, comprising: a sample receiving pad or bed, and a conjugate pad. At a minimum, there are two regions or beds. The first bed or receiving pad acts as a sponge and holds an excess of sample fluid. Once soaked, the fluid migrates from the sample receiving bed to the conjugate pad, also referred to as the test strip. This test strip contains one or more binding sites, such as surface-bound binding reagents for binding an analyte, and all the necessary components for any required detection reaction, such as a chemical reaction to occur between the target molecule, which has been immobilized, and the components within the second bed. As fluid passes, if the particular target molecule is present, a designated area on the device changes color. Typically there are at least two stripes or other shapes: one (the control) that captures any particle and thereby shows that reaction conditions and technology worked fine, the second contains a specific capture molecule and only captures those particles onto which a target analyte has been immobilized. Additionally, the membranes necessary for the lateral flow device are contained in a “housing” compartment. By “housing” compartment, it is meant that the membranes are contained in a vessel. The vessel contains one or more windows or sides that are clear, allowing for visualization of the test strip. Lateral flow devices can have quite varied structures/arrangements, and can include structures/components that can facilitate implementation of a particular assay, such as cartridges or sections that include reagents for carrying out a detection or amplification reaction. A non-limiting example of a commercially available lateral flow device is the BESt™ Cassette—Type II cassettes (Biohelix Corp, Beverly Mass., USA). BESt™ Cassette—Type II contains dried colloidal gold nanoparticles (GNPs) conjugated with anti-biotin antibody on the conjugate pad and anti-FITC and anti-dig antibodies are striped across the membrane at two lines (T) and (C) lines respectively. For detection with this device, if the specific target nucleic acid is present then the biotin tag on the amplified nucleic acid binds to the GNPs the digoxigenin tag binding to the anti-dig antibody at the C line on the membrane. Another non-limiting example of a lateral flow device is a lateral flow device available from Milenia-biotec (Germany) or custom “in-house” developed lateral flow devices with appropriate antibody (e.g., anti-digoxigenin) stripes or lines.

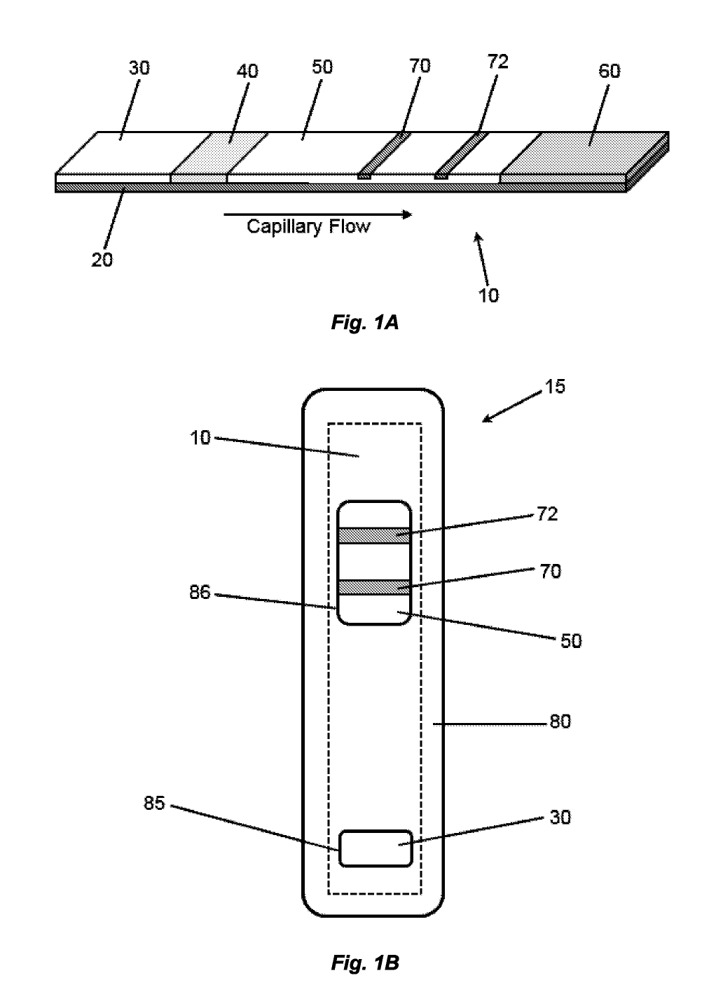

FIGS. 1A and 1B depict one aspect of a lateral flow device, with the understanding that the drawing is a schematic representation of such a device. In reference to FIGS. 1A and 1B, test strip 10 comprises a backing 20. On the backing 20 is an absorbent sample pad 30 where a sample optionally containing an analyte is deposited. In contact with sample pad 30 is a conjugate pad 40 comprising a tagged reagent for binding with the analyte to permit detection of the presence of the analyte in the sample. An example of a tagged reagent is an anti-analyte antibody (an antibody that binds the analyte), bound to a tag, such as a gold nanoparticle, a dye, a fluorophore, a quantum dot, an enzyme, or any other tag useful in this type of assay. In one aspect, the tag is biotin, a fluorophore or digoxigenin as indicated in the example below. The conjugate pad 40 is of any useful material, as are broadly known in the lateral flow device arts, that wicks fluid from the sample pad 30. A membrane 50 is provided in contact with the conjugate pad 40. The membrane 50 is of any useful material, as are broadly known in the lateral flow device arts, that wicks fluid from the conjugate pad 40. In contact with the membrane 50 is a wicking pad 60. The wicking pad 60 is of any useful material, as are broadly known in the lateral flow device arts, that wicks fluid from the membrane 50. Sample pad 30, conjugate pad 40, membrane 50 and wicking pad 60 are arranged so that liquid deposited on sample pad 40 is drawn, e.g. through capillary action, wicking, etc., through conjugate pad 40, membrane 50 and into wicking pad 60, and as such any analyte and binding reagents are drawn in a direction from the sample pad 30 to the wicking pad 60. The membrane 50 comprises a first stripe 70 and a second stripe 72, which comprise different binding reagents such that first stripe 70 comprises a binding reagent that binds complexed analyte, such that this serves as the test stripe. For example, the first stripe 70 comprises an antibody directed to the analyte, which, in the context of the present invention, an antibody or other binding reagent specific to the product produced in the presence of a target nucleic acid analyte, for example as show in the examples below. The second stripe 72 in that example would comprise a binding reagent that serves to validate the assay. As indicated in the examples below, the “test” stripe may comprise anti-FITC antibodies to bind the positive reaction products comprising FITC and biotin tags, in which case, the described gold particle-conjugated anti-biotin antibodies bind the biotin tags on the positive reaction product and co-localize at the first “test” stripe with the anti-FITC antibodies. In the examples below, an anti-digoxigenin antibody is located at the second, “control” stripe.

A novel isothermal amplification method for miRNA, or other nucleic acids, called cross hybridization amplification (CHAMP), is provided. CHAMP is a rapid, sensitive, highly specific and cost efficient alternative to current miRNA detection methods like qPCR or microarray. CHAMP could detect miR-30a-5p, with sensitivity approaching qPCR, in unprocessed urine without the need for RNA isolation or any other sample prep. Further comparative evaluation of this method will prove its utility in developing diagnostic miRNA assays.

Recently several miRNA detection systems have been reported which are based on a variety of amplification protocols, such as: PCR, padlock probes, rolling circle amplification (RCA), Invader assays, LAMP, etc. However each of these protocols have some limitations. In RCA a target miRNA functions as both the mediator for the circularization of padlock probes and the primer for RCA. However, this method requires multiple steps and discriminating a group of highly analogical miRNAs (such as the let-7 family) can be a challenging task for ligase itself. Other RCA assays have included an additional step, i.e. reverse transcription, prior to C-probe ligation and isothermal amplification to improve sensitivity, and specificity, but add to the complexity of the assays.

The detection throughput is a major concern in the quantitative analysis of miRNA profiling. Although microarray-based techniques are particularly appealing since they can examine thousands of miRNAs in parallel, their accuracy and sensitivity are still arguable. We believe that CHAMP is rapid and devoid of complexity involved in other protocols and could easily be utilized to analyze a large number of samples in a short period of time with costs approaching less than $1 per assay. It is also possible that CHAMP can also be incorporated with a multiplexed detection system, thus improving the utility of the protocol.

CHAMP was developed to quantitatively examine miRNA in a sensitive, inexpensive and isothermal amplification protocol requiring minimal sample processing. Like the TaqMan assay, this miRNA assay demonstrated sensitive and specific quantification of mature miR 30a-5p in fresh urine samples. However, instead of demanding steps of thermal cycling and expensive fluorescent probes akin to the TaqMan assay, this assay takes advantage of isothermal reaction steps and specially designed, but inexpensive primers as well as fluorescent dyes, thus enabling lower cost and greater convenience. Those features are becoming more and more important, since there are many lines of evidence showing that miRNAs might be a novel class of biomarkers for a number of conditions including cancer, transplant pathology and infectious disease diagnostics. Further studies to characterize this assay will potentially improve the availability of low cost but reliable miRNA based studies.

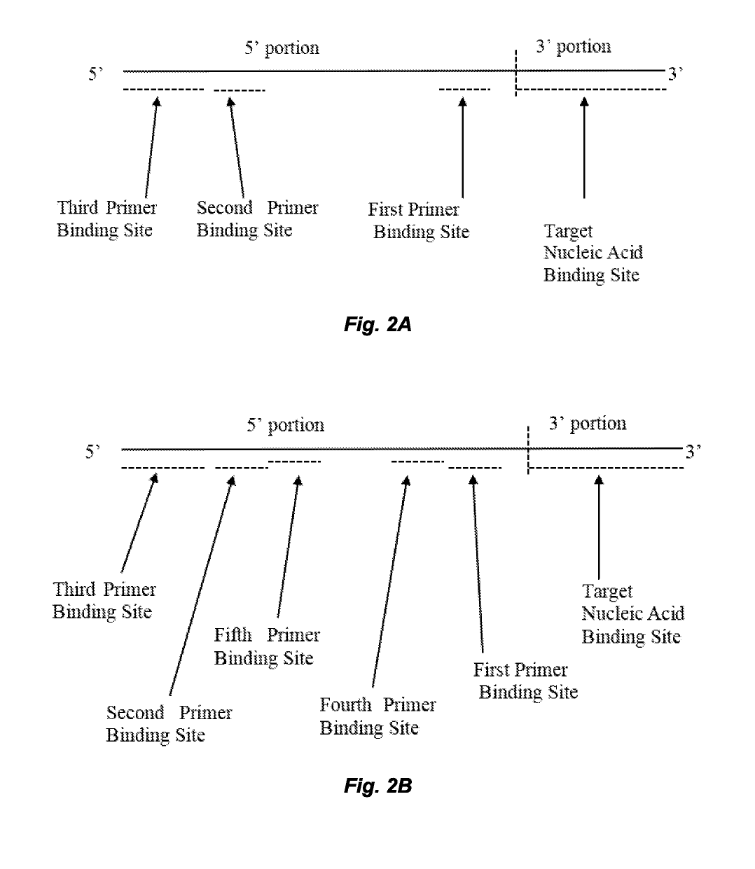

According to one aspect of the technology described herein (see, FIG. 2A), provided is a method of identifying the presence of and optionally the amount of a target nucleic acid in a sample, comprising, adding a liquid sample containing a target nucleic acid or to be analyzed for the presence of a target nucleic acid to a reaction mixture. In one embodiment, the liquid sample is blood, plasma or urine. The probe comprises a 3′ portion and a 5′ portion, wherein the 3′ portion is a sequence that binds to or is fully complementary to a target nucleic acid, and the 5′ portion comprises in a 3′ to 5′ direction a first primer binding site, a second primer binding site spaced apart from the first binding site (at least 10 bases and preferably at least 25 bases, for example 10, 15, 20, 25, 30, 35, 40, 45, 50, 60, 70, 80, 90, 100, 150, 200, 250, or 500 bases, and integers there between), and a third primer binding site. The reaction mixture comprises a first primer having a 3′ and a 5′ portion and a first tag at its 5′ end, where the 3′ portion of the first primer binds to or is fully complementary to the first primer binding site and the 5′ portion that does not bind to the probe or a sequence fully complementary to the probe. A second primer is included in the reaction mixture having a 3′ portion and a 5′ portion and a second tag, the same as or different from the first tag, at its 5′ end, where the 3′ portion of the second primer has the sequence of the second binding site or binds to a sequence fully complementary to the second binding site and the 5′ portion does not bind to a sequence fully complementary to the probe. The reaction mixture also comprises a third primer either having the sequence of the third binding site or binding a sequence complementary to the third binding site, and a third tag at its 5′ end that is different from the first and second tags. The reaction mixture also comprises suitable amounts of dNTPs, and a thermostable DNA polymerase capable of elongating from an RNA primer and having strand displacement activity and no 5′-3′ exonuclease activity, or if the target nucleic acid is DNA, a thermostable DNA polymerase having strand displacement activity and no 5′-3′ exonuclease activity. In one embodiment, at least one of the first and third tags is a ligand that can be used to immobilize the product.

According to another aspect of the technology described herein (see, FIG. 2B), provided is a method of identifying the presence of and optionally the amount of a target nucleic acid in a sample, comprising, adding a liquid sample containing a target nucleic acid or to be analyzed for the presence of a target nucleic acid to a reaction mixture. In one embodiment, the liquid sample is blood, plasma or urine. The probe comprises a 3′ portion and a 5′ portion, wherein the 3′ portion is a sequence that binds to or is fully complementary to a target nucleic acid, and the 5′ portion comprises in a 3′ to 5′ direction a first primer binding site, a second primer binding site spaced apart from the first binding site (at least 10 bases and preferably at least 25 bases, for example 10, 15, 20, 25, 30, 35, 40, 45, 50, 60, 70, 80, 90, 100, 150, 200, 250, or 500 bases, and integers there between), and a third primer binding site. The probe also has fourth and fifth primer binding sites that, in one embodiment are between the first and second binding sites, and in one embodiment, the fourth and fifth binding sites are between the first and second binding sites, and the fourth primer binding site is 3′ to the fifth primer binding site on the probe. The reaction mixture comprises a first primer having a 3′ and a 5′ portion and a first tag at its 5′ end, where the 3′ portion of the first primer binds to or is fully complementary to the first primer binding site and the 5′ portion has the sequence of the fourth primer binding site or binds to a sequence fully complementary to the fourth binding site. A second primer is included in the reaction mixture having a 3′ portion and a 5′ portion and a second tag, that is the same as or different from the first tag, at its 5′ end, where the 3′ portion of the second primer has the sequence of the second binding site or binds a sequence fully complementary to the second binding site and the 5′ portion binds to or is fully complementary to the fifth binding site. The reaction mixture also comprises a third primer having the sequence of the third binding site or binding a sequence fully complementary to the third binding site and a third tag at its 5′ end that is different from the first and second tags. The reaction mixture also comprises suitable amounts of dNTPs, and a thermostable DNA polymerase capable of elongating from an RNA primer and having strand displacement activity and no 5′-3′ exonuclease activity, or if the target nucleic acid is DNA, a thermostable DNA polymerase having strand displacement activity and no 5′-3′ exonuclease activity. In one embodiment, at least one of the first and third tags is a ligand that can be used to immobilize the product.

The reaction mixture is incubated at a temperature effective to produce reaction product from the DNA polymerase in the presence of the target nucleic acid. The method further comprises determining the presence of, and optionally the quantity of the target nucleic acid in the sample by detecting nucleic acids larger than the first and second primer tagged with the first and second tags. The presence of a target nucleic acid in the sample can be determined by any method, such as, without limitation by lateral flow assay or agarose gel electrophoresis, for example as shown below. Quantification of the target nucleic acid typically is accomplished by more sophisticated methods, such as by comparison of the rate of accumulation of product from a sample, to the rate of accumulation of product of known concentration in a control reaction. Such comparisons typically are automated. Examples of graphs showing accumulation of product are shown in the Examples below.

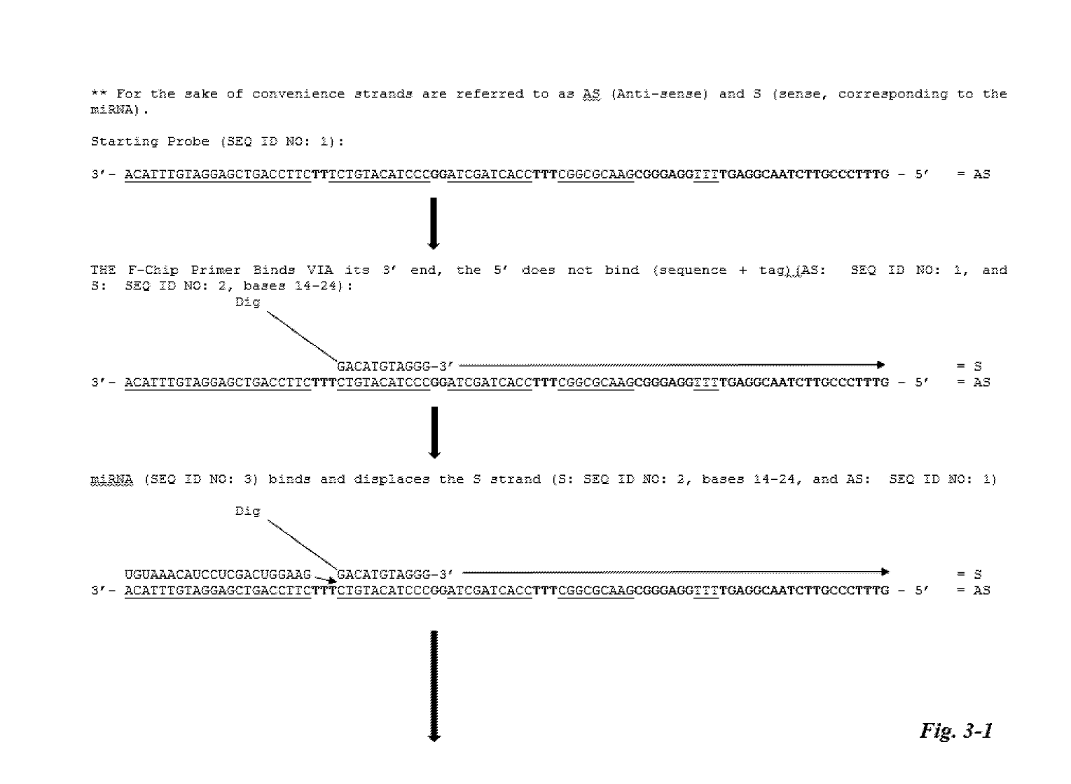

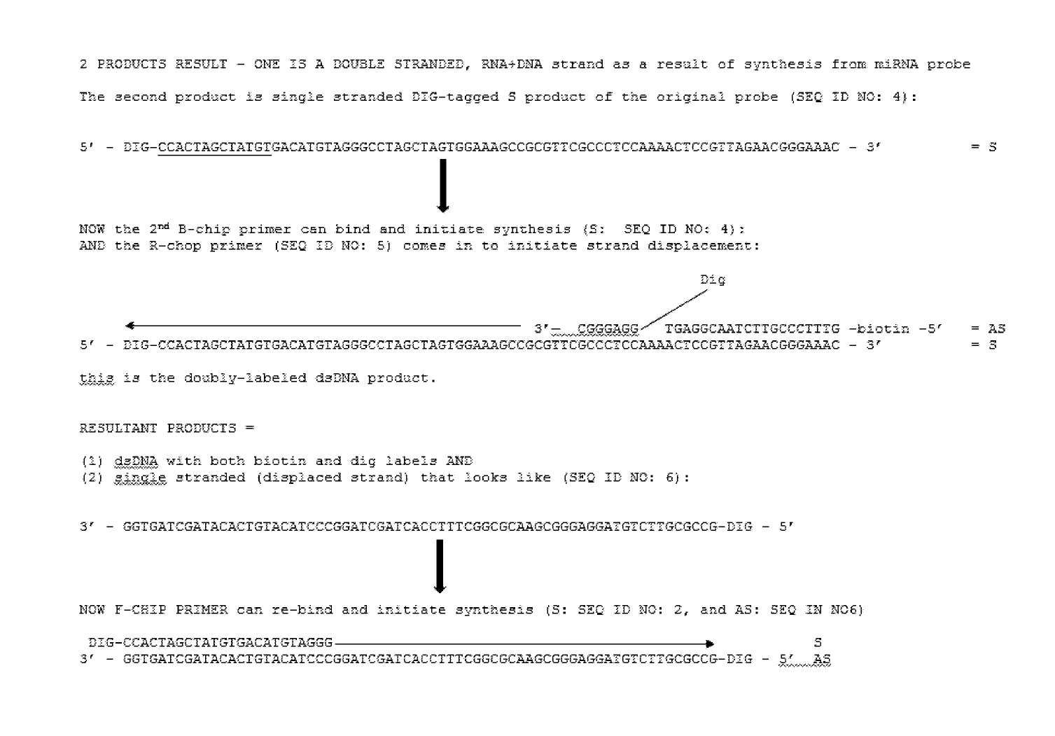

This method of identifying the presence of and optionally the amount of a target nucleic acid in a sample, as mentioned above, is referred to herein as cross hybridization amplification (“CHAMP”). The method is an isothermal amplification reaction, which amplifies the target nucleic acid sequences at a constant temperature, through the utilization of a probe, three or more primers and a thermostable DNA polymerase capable of elongating from an RNA primer (when the target nucleic acid is an RNA) and having strand displacement activity and no 5′-3′ exonuclease activity. CHAMP functions at a constant temperature, eliminating the need for expensive thermocyclers. In CHAMP, a single-stranded, nucleic acid probe is used to bind to a target specific target nucleic acid sequences, having a 3′ portion that binds specifically to the target nucleic acid, such as a miRNA, and a 5′ portion that is amplified in the presence of the target nucleic acid. The same 5′ portion can be used for detection of different target nucleic acids, and thus can be, in one method, attached to a sequence complementary to the target nucleic acid (the 3′ portion). The target sequence can be DNA or RNA. A non-limiting example of the process of CHAMP that is shown in the Examples below, is believed to function, at least in part, by the mechanism shown in FIG. 3. The first primer sequence, having a 3′ end portion complementary to a target sequence of the probe, a 5′ portion that does not bind to the probe, e.g., having a sequence within the probe, and a first tag at its 5′ end, binds to the original probe sequence by its 3′ portion. This binding location of the 1^{st} primer, on the probe, is 5′ to the miRNA recognition site. Through the functionality of a thermo-stable polymerase lacking 5′→3′ exonuclease activity, such as Bst or Pyrophage, an antisense strand is generated from the first probe. The target nucleic acid binds 3′ to the first primer on the probe and then is elongated by the strand-displacing polymerase, thereby displacing the strand primed by the first primer. The chosen thermo-stable polymerase lacking 5′→3′ exonuclease activity, mentioned above, is additionally able to synthesis a complimentary sequence from the target nucleic acid (e.g., miRNA), acting as a primer, allowing for strand displacement. It should be noted that when the target nucleic acid is RNA, the DNA polymerase is required to be able to elongate from an RNA primer on a DNA template. Bst, Large Fragment and Pyrophage have this activity. If the target nucleic acid is DNA, the polymerase does not need the RNA-priming capability. The second primer comprises a 3′ portion, a 5′ portion, and a second tag, that is the same as the first tag or different from the first tag, at its 5′ end, where the 3′ portion of the second primer has a sequence that is present in the probe and therefore binds the displaced product of the first primer at a second binding site of the probe, while the 5′ portion of the second primer that is tagged with the second tag does not bind the displaced product of the first primer, but optionally will bind to the probe. The polymerase then synthesizes a sequence that, like the probe, is complementary to the product containing the first primer, in other words the product containing the second primer has at least a portion of the sequence of the probe. A third primer comprises a third tag (biotin) at its 5′ end that is different from the first and second tag and has a sequence found 5′ to the binding site of the second primer on to the probe, binds to the product of the first primer. The polymerase elongates from the third primer, thereby displacing the product of the second primer, resulting in a double-stranded product that is Dig-tagged at one end and biotin-labeled at the other. Although the described reaction is a principle reaction that is thought to occur, the optional presence of the 5′ portions of the first and second primers that are of opposite polarity to the 3′ portion (that is, complementary to or binding to the opposite strand as the 3′ ends of the same primer, or sense versus antisense) are thought to facilitate creation of secondary products of varying length, resulting in the ladder or smear seen in the agarose gels described below in the Examples.

The reaction mixture contains the target nucleic acid, or is tested to determine if it contains the target nucleic acid. In one example, the sample is an un-processed sample of a bodily fluid from a patient, such as a human patient. Examples of bodily fluids include blood, plasma, serum, saliva, cerebrospinal fluid, lymph, semen, or urine. The reaction mixture comprises the three or more primers as discussed above, the probe and the other necessary components, e.g., as outlined above required for the reaction to occur. The other components of the reaction mix are as defined above. The reaction mixture can then be incubated, for example, between 50-70 degrees (for example, 50, 51, 52, 53, 54, 55, 56, 56, 57, 58, 59, 60, 61, 62, 63, 64, 65, 66, 67, 68, 69, 70, including all increments in between) for 30-90 minutes (for example, 30, 35, 40, 45, 50, 55, 60, 65, 70, and all increments in between). Exceptions to these limits of temperature and duration may exist. In one embodiment, the reaction is incubated at 65° C. for 60 minutes when Bst, large Fragment is the chosen thermostable polymerase. For every primer, probe, target sequence and polymerase combination, the optimal concentration of respective ingredients, and temperature and duration of reaction can be optimized by a person of ordinary skill. After sufficient time has been provided for the reaction to occur, the reaction mixture is then analyzed for the presence of a reaction product that is only present when the reaction mixture contains the target nucleic acid. In any detection method, a test sample can be compared to appropriate positive and/or negative controls.

In certain aspects, the presence of the reaction product is determined by gel electrophoresis, free-flow electrophoresis, or in a lateral flow device, real time isothermal or PCR devices. Such methods are broadly known, including numerous, if not a multitude of, variations on those methods. One of ordinary skill can determine the presence of and/or quantify the reaction product of the amplification reactions described herein. The detection methods described herein are merely exemplary of the many different methods available to those of ordinary skill to detect the presence of and/or quantify a specific nucleic acid in a sample. In one aspect, the reaction is resolved on a 2% agarose gel containing an appropriate amount of ethidium bromide for one hour at 100 volts. The agarose gel may contain anywhere 1% between 2% of agarose. Upon electrophoresis, presence of product is determined through ultra-violet detection for nucleic acid. Presence of the target nucleic acid sequence will appear as a smear or ladder. The reaction product can further be blotted, and the specific tag present on the reaction product can be specifically detected.

In another aspect, a method is provided for rapid in-situ hybridization detection of a target nucleic acid sequence. The currently-prevailing mode of analysis and detection of small RNA molecules and/or other types of nucleic acids, such as miRNAs, in frozen sections of pathological samples is through, Locked-nucleic acid-in-situ hybridization. (LNA-ISH). LNA-ISH is an extremely complicated, time-consuming, and error-prone process. LNA-ISH protocol typically takes up to 7 days to complete. As such, provided herein is an in situ method that utilizes the assays described herein to reduce this time frame to 3 days or less, while maintaining improved specificity and sensitivity. Specifically, the first two primers, as defined above, are tagged with any suitable in-situ hybridization label, such as Digoxigenin. Detection of a specific target nucleic acid sequence is achieved by first obtaining tissue samples on a glass slide that have been obtained by cryosectioning and permeabilizing. This is followed by first incubating these samples with the target-specific probe for several (e.g., 6-12) hours followed by a washing step. Next, the samples are incubated with all other necessary components of the reaction mixture, as defined above (e.g., Bst polymerase, primers, etc.), required for the reaction to occur. The samples are incubated with the reaction mixture at the appropriate temperature and time, as outlined above. For example, a non-limiting example would be at 65° C. for 60 minutes. If the specific target nucleic acid sequence is present in the sample, then the reaction occurs. The sample is then incubated with an AP-conjugated antibody directed towards the tag (such as an anti-Digoxigenin-AP antibody). Subsequently, the sample is incubated with a chromogenic substrate, such as BM purple (Roche), and the alkaline phosphatase conjugated to the anti-DIG antibody will remove a phosphate group from the substrate, and a dark purple chromogenic precipitate will form in the sample at sites that contain the nucleic acid target. Presence of this purple color, and its intensity, can function as a semi-quantitative measure of the presence of the target nucleic acid.

According to another non-limiting embodiment, of the technology described herein, provided is a kit for the detection of a specific target nucleic acid sequence. The kit contains the described primers and probes according to any embodiment described herein, and optionally a suitable polymerase as described above, a sample of the target sequence for use in a control experiment, a reaction mixture comprising additional reagents necessary to conduct the reaction, such as dNTPs, buffers, salts, MgSO₄, reducing agents, etc. The reaction mixture comprises suitable amounts of the probes and primers, e.g., in a single, one-assay vessel. The kit may comprise any additional reagents or apparatus to facilitate the reaction. As indicated below, the reaction can be conducted using urine without processing. However, for sources other than urine, such as blood, filters, centrifuge tubes, affinity columns or the like, etc. can be provided in the kit. The liquid or lyophilized elements of the kit are typically packaged in vessels, such as tubes, or other containers for storing and transporting such materials, and/or for conducting a reaction. The kit also may comprise indicia, such as a tag or instructional booklet describing at least the methods for conducting the reaction, essentially as described herein.

In one example, the kit comprises: (a) a probe comprising a 3′ portion and a 5′ portion, wherein the 3′ portion is a sequence that binds to or is fully complementary to a target nucleic acid, and the 5′ portion comprises in a 3′ to 5′ direction in reference to the probe, a first primer binding site, a second primer binding site spaced apart from the first binding site (at least 10 bases and preferably at least 25 bases, for example 10, 15, 20, 25, 30, 35, 40, 45, 50, 60, 70, 80, 90, 100, 150, 200, 250, or 500 bases, and integers there between), and a third primer binding site, and further comprising fourth and fifth primer binding sites that, in one aspect are between the second and third binding sites, and in one aspect, the fourth primer binding site is 5′ to the fifth primer binding site; (b) a first primer having a 3′ and a 5′ portion and a first tag at its 5′ end, where the 3′ portion of the first primer binds to or is fully complementary to the first primer binding site and optionally, the 5′ portion has the sequence of the fourth primer binding site or binds to a sequence fully complementary to the fourth binding site; (c) a second primer is included in the reaction mixture having a 3′ portion and a 5′ portion and a second tag, that is the same as or different from the first tag, at its 5′ end, where the 3′ portion of the second primer has the sequence of the second binding site or binds a sequence fully complementary to the second binding site and optionally, the 5′ portion binds to or is fully complementary to the fifth binding site; and (d) a third primer having the sequence of the third binding site or binding a sequence fully complementary to the third binding site and a third tag at its 5′ end that is different from the first and second tags. In one embodiment, at least one of the first and third tags is a ligand that can be used to immobilize the product.

In another embodiment, the kit comprises: (a) a probe comprising a 3′ portion and a 5′ portion, wherein the 3′ portion is a sequence that binds to or is fully complementary to a target nucleic acid, and the 5′ portion comprises in a 3′ to 5′ direction a first primer binding site, a second primer binding site spaced apart from the first binding site (at least 10 bases and preferably at least 25 bases, for example 10, 15, 20, 25, 30, 35, 40, 45, 50, 60, 70, 80, 90, 100, 150, 200, 250, or 500 bases, and integers there between), and a third primer binding site; (b) a first primer having a 3′ and a 5′ portion and a first tag at its 5′ end, where the 3′ portion of the first primer binds to or is fully complementary to the first primer binding site and the 5′ portion that does not bind to the probe or to a sequence fully complementary to the probe; (c) a second primer having a 3′ portion and a 5′ portion and a second tag, the same as or different from the first tag, at its 5′ end, where the 3′ portion of the second primer has the sequence of the second binding site or binds to a sequence fully complementary to the second binding site and the 5′ portion does not bind to the probe or to a sequence fully complementary to the probe; and (d) a third primer either having the sequence of the third binding site or binding a sequence complementary to the third binding site, and a third tag at its 5′ end that is different from the first and second tags. In one embodiment, at least one of the first and third tags is a ligand that can be used to immobilize the product.

In one embodiment, the kit comprises one vessel containing the reaction mixture, as described above, including the polymerase, but excluding the primers and the probe. A second vessel will comprise a mixture of the primers and a third vessel will comprise a probe specific to the target nucleic acid. The primers and probe of the second and third vessels may be in liquid form or lyophilized. Lyophilization would help ensure longevity, and prevent any chance of degradation, of the primers and probe. This kit can then be used, for example, in combination with a lateral flow device, for example as described herein, for determining if a specific target nucleic acid sequence is present in a given biological sample.

According to another non-limiting embodiment, of the technology described herein, provided is a kit allowing for high-throughput screening of any given target nucleic acid sequence of choice. In one embodiment, the kit is in the form of a specialized cartridge adapted for a specific high-throughput robotics/fluidics system, for example as described above. The various elements of the reaction can be packaged in the kit or cartridge separately or together, or in multiple cartridges or vessels. Using an automated system, a high-throughput screening of patients can then be performed. Similar to a tissue microarray technique, a hollow needle can be used to remove tissue cores (for example from kidney, or any other appropriate tissue/organ), from several hundreds to thousands of patients, as small as 0.6 mm in diameter from regions of interest. These tissue cores are then inserted in a recipient paraffin block in a precisely spaced, array pattern. Sections from this block are cut, mounted on a microscope slide and then analyzed by any method of standard histological analysis, such as but not limited to the in-situ hybridization method described above.

Additionally, and similarly to the high-throughput method described above. Another non-limiting embodiment of the technology disclosed herein, is a kit, as described above. A high-throughput robotics system, not limited to, but such as, the Perkin Elmer FlexDrop, can then be utilized to screen several hundreds to thousands of patients. Diagnostic plates, such as but not limited to, 96-well plates, are loaded with patient samples, such as urine or blood. These plates are placed on a heating mechanism that keeps the plates at the required reaction temperature, for example, 65° C. The high-throughput robotics system can then automate the rest of the process by pipetting/injecting the appropriate amount of probe, primers, and reaction mixture to each biological sample. Once the biological samples have received the appropriate amount of probe, primers, and reaction mixture, the entire sample reaction incubates for the appropriate time, such as 60 minutes. Once the reaction has completed, the sample reactions can then be processed through gel electrophoresis or any equivalent assay that will detect the presence or absence of the target nucleic acid sequence.

### EXAMPLES

The present invention will be described in part in the following Example(s). However, the invention should not be considered so limited, and should instead be defined by the appended claims.

### Example 1

### Materials and Methods:

Mature miRNA, precursor miRNAs sequences: Sequences of mature and miRNAs miR 30a -5p were selected from the Sanger Center miRbase, and from the NCBI database. Synthetic mature miRNAs were purchased from IDT (Coralville, Iowa).

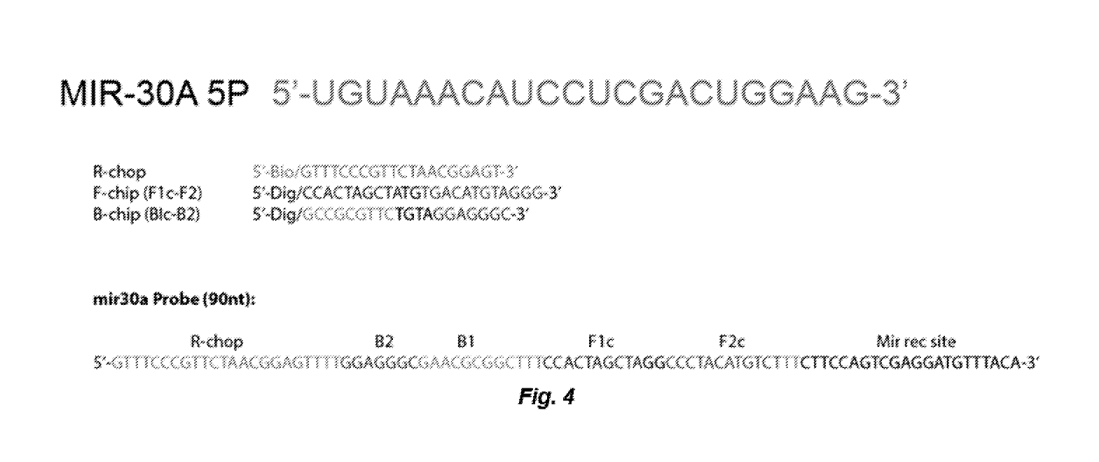

Design of CHAMP primers: We designed 20-25 nt isothermal primers (F-Chip, B-Chip, R-Chop) and a probe specific for miR-30a-5p (FIG. 4). The primers had a binding site for mature miR-30a-5p via a 20 nt sequence at the 3′-end, and were tagged with either biotin or digoxigenin on the 5′-end. All primers were synthesized by IDT (Coralville, Iowa).

Tissue and sample preparation and total RNA extraction: Dissected embryonic mouse kidneys, and de-identified urine samples were utilized for the preparation of total RNA samples using RNeasy kit and reagents (Qiagen) following the manufacturer's procedure. All animal procedures were in accordance with Institutional Animal Care and Use Committee (IACUC) guidelines. Nucleic acid concentrations were determined by absorption readings at 260 nm on a Spectrophotometer (BioRad, SmartSpec).

CHAMP reactions: CHAMP reactions were performed in 25 μL volume using synthetic miRNA 30a-5p and urine samples in this study. Urine samples were spiked with synthetic miRNA 30a-5p. A number of concentrations were used, ranging from 10000 to 1000000 copies, and samples were assayed both with and without RNA extraction. RNA was extracted with Qiagen RNAeasy kit. Five μL of unprocessed urine or extracted RNA from 200 uL of urine was used as target for the CHAMP reaction.

Optimization of CHAMP reactions: To optimize the reactions for CHAMP, real time assays and gel electrophoresis was performed using an Eva Green containing mastermix. We optimized for temperature, magnesium concentration, and Bst DNA polymerase enzyme concentration. We also optimized for different dilutions of urine. A 10-fold dilution series of synthetic miRNAs were used to detect the sensitivity of the assay.

CHAMP reaction setup: The total reaction volume of 25 μL contained, the three primers and the probe. The other components of the reaction mix were: 2.5 μL of 10× Bst DNA polymerase reaction buffer, 2 μL of 8 U/μL Bst DNA polymerase (New England Biolabs Inc., MA, USA), 2 μL of 100 mM of MgSO₄, 5 μL of Betaine (Sigma Aldrich, MO, USA), 2.5 μL deionized water and 2.5 μL of target sample. The Bst DNA polymerase reaction buffer (1×) contains 20 mM Tris-HCl (pH 8.8, @ 25° C.), 10 mM KCl, 10 mM (NH₂)SO₄, 2 mM MgSO₄, 0.1% Triton X-100 (New England Biolabs Inc., MA, USA). The reaction mixture was incubated at temperature of 64° C. for 60 minutes. The CHAMP reaction was carried out in a heating block (LAB-LINE, Barnstead International, Dubuque, Iowa, USA) as well as the real time PCR machine. The negative control contained all the reaction components listed above, except for the respective template (NTC). The CHAMP amplification products were detected by in real time as well as by gel electrophoresis and on LFD.

Real-time CHAMP assay: The isothermal amplification was performed in a 25-μL volume containing 1 μL of probe, 1.2 μM of each primer. The reactions were incubated at 65° C. in a 96-well plate and were real-time monitored for 60 minutes by an Opticon2 DNA Engine (Bio-Rad). All reactions were run in duplicate. The threshold cycle (CT) was defined as the fractional cycle number at which the fluorescence passed the fixed threshold. Real-time CHAMP assay was compared with miRNA real time PCR assay.

miRNA real-time PCR assay: To quantify the miRNA expression level by the real-time PCR method, the cDNA was synthesized using the miRNA cDNA Synthesis Kit (Invitrogen). The resulting cDNA was subjected to real-time PCR using qPCR primers, probe and Mix (Life Technologies). Quantitative Real-time PCR was used under the following thermo cycler conditions: 50° C. for 2 mM, 95° C. for 2 mM, 40 cycles of 95° C. for 15 sec, and 60° C. for 50 sec.

Scheme of Detection by LFD: Commercially available LFDs, BESt™ Cassette—Type II cassettes (Biohelix Corp, MA, USA) were used to detect the miR-30a-5p CHAMP products. The BESt™ Cassette—Type II cassette contains dried colloidal gold nanoparticles (GNPs) conjugated with anti-biotin antibody on the conjugate pad and anti-FITC and anti-dig antibodies are striped across the membrane at two lines (T) and (C) lines respectively. Approximately 10 μL of the CHAMP amplification product was used for detection in each BESt™ Cassette—Type II cassette and the reaction allowed to develop for 5 minutes. For detection with this LFD, the amplicons, along with the lateral flow buffer was applied to the sample pad. The biotin tag on the amplified DNA binds to the GNPs present on the conjugate pad and proceeds to flow on to the nitrocellulose membrane. If amplicon was present, its detection was based on the dig tag binding to the anti-dig antibody at the C line on the membrane.

### Results:

We developed the CHAMP process utilizing miR 30a-5p as the target miRNA. This CHAMP miRNA assay consisted of a single step process. Amplification required the binding of the primers and miRNA to the probe, DNA synthesis and strand displacement by Bst DNA polymerase enzyme. The reactions required a simple isothermal incubation at 65° C. for 30-60 minutes. Assay optimization was performed on water as well as fresh urine samples spiked with synthetic miR-30a-5p target. Further details are presented below.

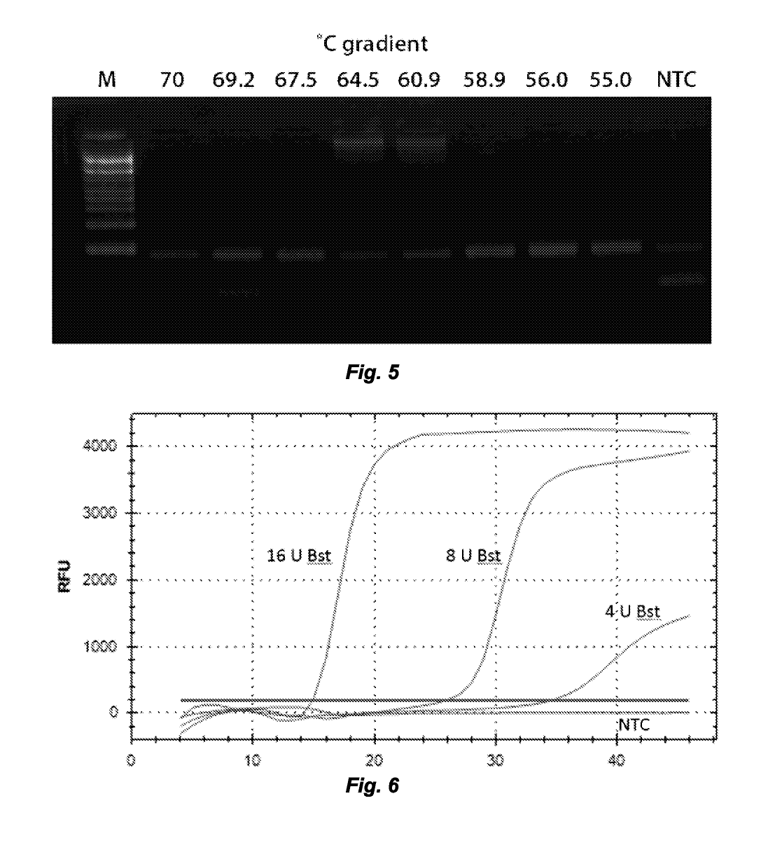

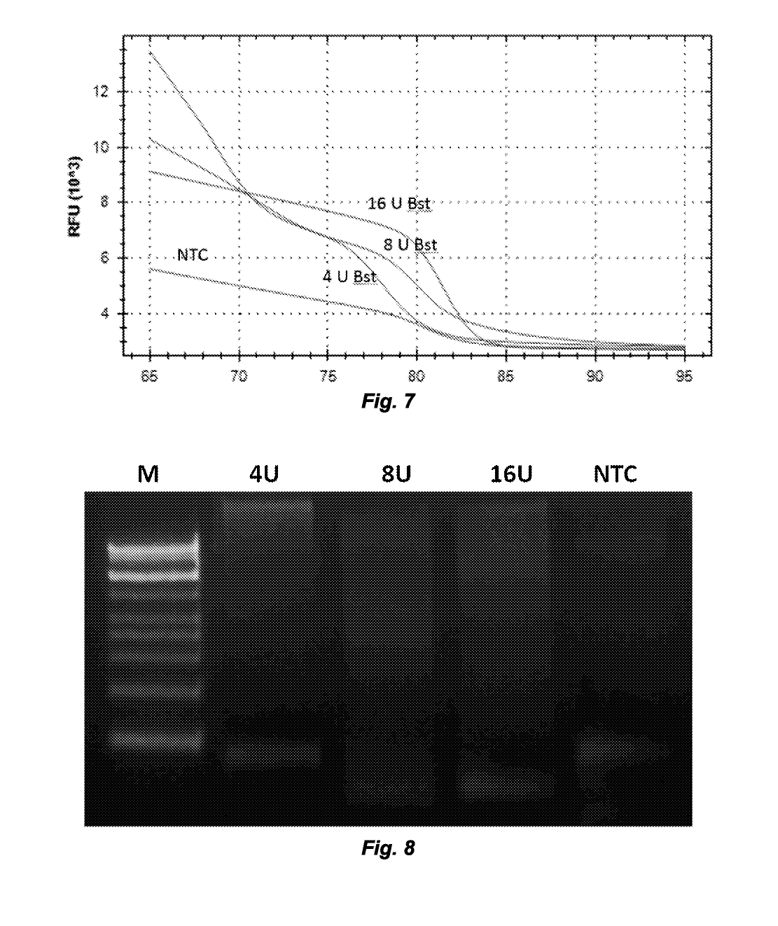

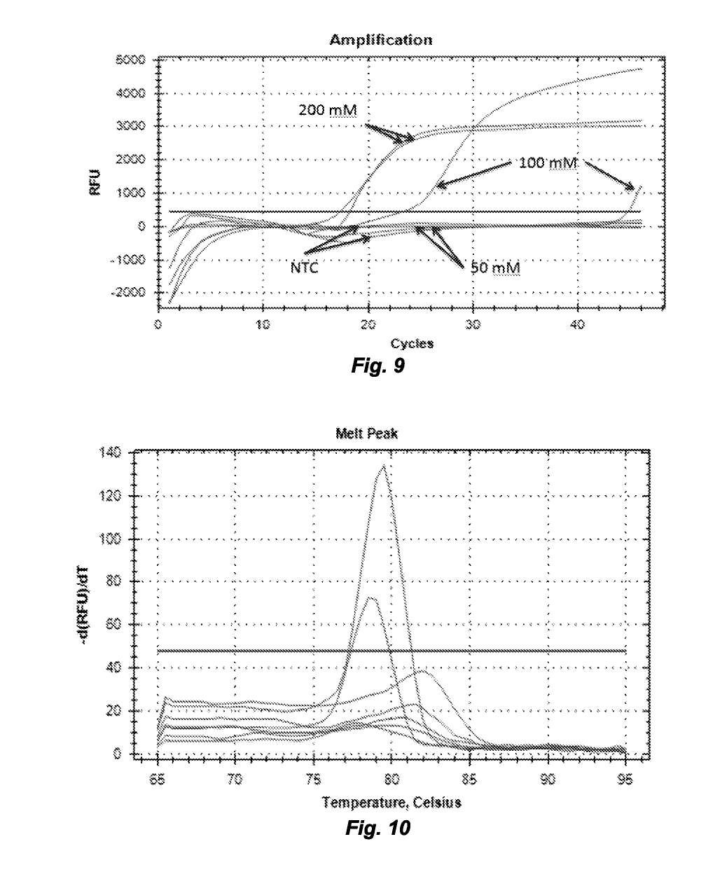

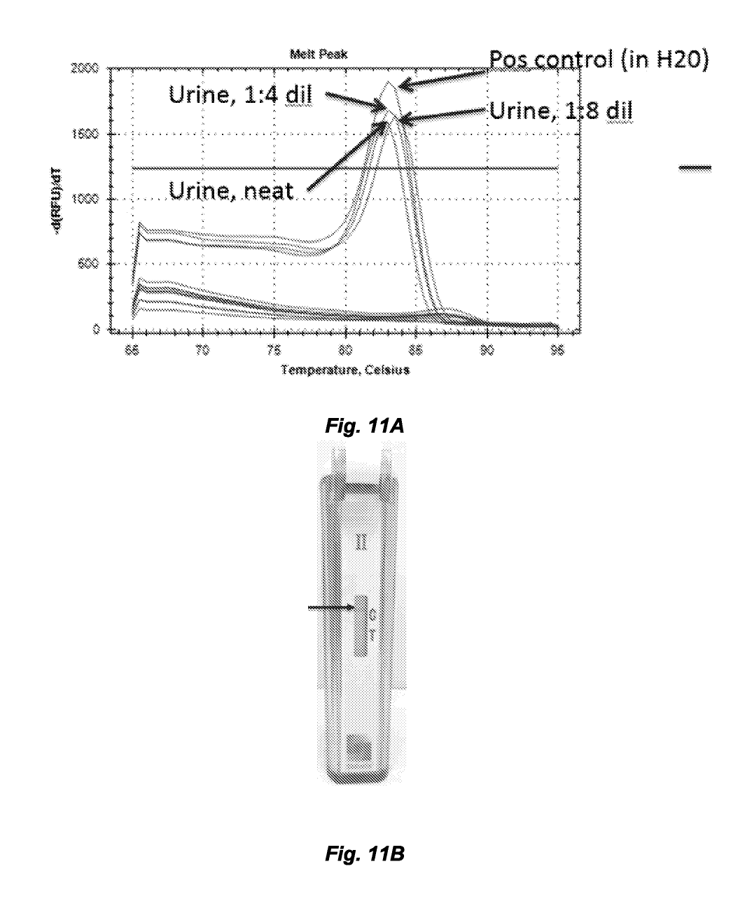

A schematic description of the CHAMP miRNA assay is shown in FIG. 3. The results of assay optimization are shown in FIGS. 4-8. The CHAMP assay performed best at 64.5° C., with 16 units of Bst DNA polymerase and 200 mM of MgSO₄ concentration. Using the same assay conditions, the amplification and melting curves for miR-30a were obtained within 1 hr (<20 min in FIG. 6 using 16 units Bst polymerase). Amplicons were also detected by UV trans-illumination and on agarose gels (FIGS. 5 and 8). The optimized conditions were used to determine the sensitivity of the assay which was found to be 20 copies (FIG. 10). The presence of urine did not inhibit the reaction, and unprocessed urine spiked with synthetic miR-30a could be used in the reactions, without the need for RNA isolation (FIG. 11a). The incorporation of biotin and digoxigenin in the primers allowed for the detection of amplicons on lateral flow strips (FIG. 11b).

Development of In Situ Hybridization (ISH) Detection of miRNA using CHAMP Isothermal Amplification.

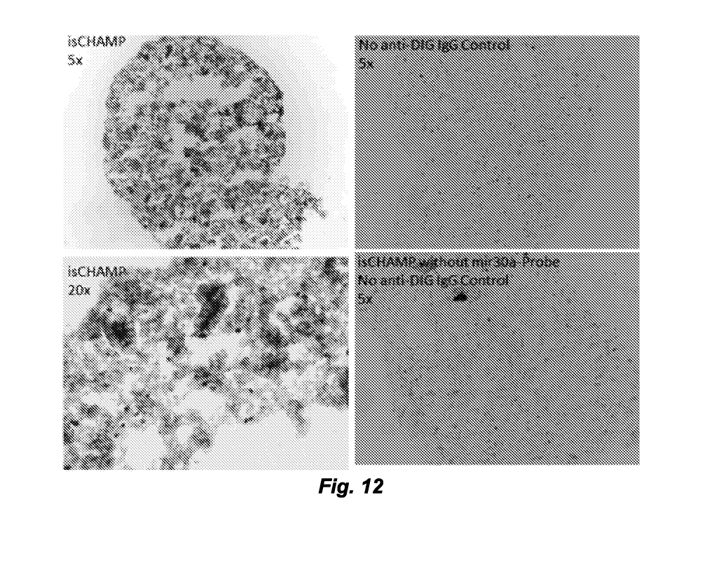

The currently prevailing mode of analysis by LNA-ISH is an extremely complicated, time-consuming, and error-prone process. In our prior experience, the LNA-ISH protocol typically takes up to 7 days to complete. We developed in situ-CHAMP for the detection of miR-30a-5p, using the CHAMP assays described above to reduce this time frame to 3 days of less while maintaining improving specificity and sensitivity. For these reactions, the F-Chip and B-Chip primers are tagged with DIG, and amplified targets are visualized by incubating slides of embryonic kidney sections with AP-conjugated antibody and a color reaction development with BM purple (FIG. 12).

### Example 2

Sequences of mature and miRNAs miR-30a-5p (miRBase MIMAT0000087, 5′-UGUAAACAUCCUCGACUGGAAG-3′ (SEQ ID NO: 3)), miR-30b (miRBase MIMAT0000420, 5′-UGUAAACAUCCUACACUCAGCU-3′ (SEQ ID NO: 8)), and miR-let-7 (miRBase MIMAT0000064 5′-UGAGGUAGUAGGUUGUAUGGUU-3′ (SEQ ID NO: 9)) were selected online from the Sanger Center miRbase, and the NCBI database. Synthetic miRNAs were purchased from Integrated DNA Technologies (IDT, Coralville, Iowa).

Isothermal amplification reactions were conducted using the miRNA 30a-5p probe and primer as indicated in Example 1. Normal, unprocessed urine was spiked with 1 μg of either miR-30a-5p, miR-30b, or miR-let-7 synthetic RNA. Amplification product was identified using gel and real time PCR. Target nucleic acid analyte miR-30a-5p was detected. Closely-related miR-30b, and miR-let-7 were not detected.

### Example 3

The detection limit of the assay was determined. Isothermal amplification reactions were conducted using the miRNA 30a-5p probe and primer as indicated in Example 1. Serial dilutions of synthetic miR30a-5p in water were tested for limit of detection of the assay. Amplification product was identified using gel, LFD, and real time PCR analyses. The detection limit for the target nucleic acid analyte, miR-30a-5p, was 20 copies in water.

### Example 3

The ability of the assay to identify urine from patients having nephrotic syndrome (NS) was determined. Isothermal amplification reactions were conducted using the miRNA 30a-5p probe and primer as indicated in Example 1. Samples of normal unprocessed urine (n=6) and de-identified, unprocessed urine from clinical NS patients (n=5) were tested. Amplification product was identified using gel, LFD, and real time PCR analyses. The quantitative results were obtained by real time analyses. Analyses of patient samples showed an 8- to 200-fold increase in miRNA in urine of patients with NS (n=5) vs controls (n=6).

Having described this invention, it will be understood to those of ordinary skill in the art that the same can be performed within a wide and equivalent range of conditions, formulations and other parameters without affecting the scope of the invention or any embodiment thereof.

1. A method of identifying the presence of and/or the amount of a target nucleic acid in a sample, comprising:
a. adding a sample to a reaction mixture comprising:
i. a thermostable DNA polymerase having strand displacement activity and no 5′-3′ exonuclease activity;
ii. deoxyribonucleotide triphosphates;
iii. a nucleic acid or nucleic acid analog probe comprising a 3′ portion and a 5′ portion, wherein the 3′ portion is a sequence that binds to or is fully complementary to the target nucleic acid, and the 5′ portion comprises in a 3′ to 5′ direction a first primer binding site, a second primer binding site spaced apart from the first binding site, and a third primer binding site;
iv. a first primer having a 3′ portion and a 5′ portion and a first tag at its 5′ end, where the 3′ portion of the first primer binds to or is fully complementary to the first primer binding site and the 5′ portion does not bind to the probe;
v. a second primer having a 3′ portion and a 5′ portion and a second tag, the same as or different from the first tag, at its 5′ end, where the 3′ portion of the second primer has the sequence of the second binding site or binds to a sequence fully complementary to the second binding site and the 5′ portion does not bind to a sequence fully complementary to the probe; and
vi. a third primer either having the sequence of the third binding site or binding a sequence complementary to the third binding site, and a third tag at its 5′ end that is different from the first and second tags;
b. incubating the reaction mixture at a temperature effective to produce reaction product from the polymerase in the presence of the target nucleic acid; and
c. determining the presence of and/or quantifying production of reaction product in the sample by detecting and/or quantifying nucleic acids larger than the first and second primer tagged with the first and second tags.

2. The method of claim 1, wherein the probe further comprises:
a. one or both of a fourth and fifth primer binding site; and
b. the 5′ portion of the first primer has the sequence of the fourth primer binding site or binds to a sequence fully complementary to the fourth binding site and/or the 5′ portion of the second primer binds to or is fully complementary to the fifth binding site.

3. The method of claim 1, wherein, the target nucleic acid is an RNA, and the thermostable DNA polymerase having strand displacement activity and no 5′-3′ exonuclease activity and the thermostable DNA polymerase with no 5′-3′ exonuclease activity can elongate from an RNA primer.

4. The method of claim 1, wherein the sample is a biological sample.

5. The method of claim 4, wherein the biological sample is a bodily fluid.

6. The method of claim 5, wherein the biological sample is urine or blood.

7. (canceled)

8. The method of claim 4, wherein the 5′ portion of the probe does not bind a nucleic acid of the biological sample and/or of the organism from which the biological sample is obtained.

9. The method of claim 1, wherein, the temperature effective to produce reaction product is from 60° C. to 70° C.

10. The method of claim 1, wherein, the target nucleic acid is detected by gel electrophoresis.

11. The method of claim 1, wherein, the sample is an organ, a tissue sample or a section of a tissue sample on a slide and the reaction mixture is applied to the sample and is incubated and detected in-situ.

12. (canceled)

13. (canceled)

14. (canceled)

15. The method of claim 1, in which the 5′ portion has the sequence:
(SEQ ID NO: 1, bases 1-68)
5′-GTTTCCCGTT CTAACGGAGT TTTGGAGGGC GAACGCGGCT
TTCCACTAGC TAGGCCCTAC ATGTCTTT 3′;
and the first, second and third primers have the sequences, respectively:
(SEQ ID NO: 2)
5′-CCACTAGCTA TGTGACATGT AGGG-3′,
(SEQ ID NO: 7)
5′-GCCGCGTTCT GTAGGAGGGC-3′,
and
(SEQ ID NO: 5)
5′-GTTTCCCGTTCTAACGGAGT-3′.

16. The method of claim 1, in which the 3′ portion has the sequence:
(SEQ ID NO: 1, bases 69-90)
5′-CTTCCAGTCGAGGATGTTTACA-3′.

17. The method of claim 1, in which the probe has the sequence:
(SEQ ID NO: 1)
5′-GTTTCCCGTT CTAACGGAGT TTTGGAGGGC GAACGCGGCT
TTCCACTAGC TAGGCCCTAC ATGTCTTTCT TCCAGTCGAG
GATGTTTACA-3′;
and the first, second and third primers have the sequences, respectively:
(SEQ ID NO: 2)
5′ CCACTAGCTATGTGACATGTAGGG-3′,
(SEQ ID NO: 7)
5′ GCCGCGTTCTGTAGGAGGGC 3′,
and
(SEQ ID NO: 5)
5′ GTTTCCCGTTCTAACGGAGT 3′.

18. A kit comprising one or more one vessels, or a cartridge comprising compartments, comprising:
i. a nucleic acid or nucleic acid analog probe comprising a 3′ portion and a 5′ portion, wherein the 3′ portion is a sequence that binds to or is fully complementary to the target nucleic acid, and the 5′ portion comprises in a 3′ to 5′ direction a first primer binding site, a second primer binding site spaced apart from the first binding site, and a third primer binding site;
ii. a first primer having a 3′ portion and a 5′ portion and a first tag at its 5′ end, where the 3′ portion of the first primer binds to or is fully complementary to the first primer binding site and the 5′ portion does not bind to the probe;
iii. a second primer having a 3′ portion and a 5′ portion and a second tag, the same as or different from the first tag, at its 5′ end, where the 3′ portion of the second primer has the sequence of the second binding site or binds to a sequence fully complementary to the second binding site and the 5′ portion does not bind to a sequence fully complcmentary to the probe; and a third primer either having the sequence of the third binding site or binding a sequence complementary to the third binding site, and a third tag at its 5′ end that is different from the first and second tags.

19. The kit of claim 18, further comprising:
a. a reaction mixture comprising:
i. a thermostable DNA polymerase having strand displacement activity and no 5′-3′ exonuclease activity, and optionally being capable of elongating from an RNA primer; and
ii. deoxyribonucleotide triphosphates.

20. The kit of claim 18, further comprising a lateral flow device for detecting product of an isothermal reaction that proceeds in the presence of the target nucleic acid.

21. (canceled)

22. The kit of claim 18, further comprising an agarose or acrylamide electrophoresis gel for use in detection of products of a reaction using the reaction mixture.

23. (canceled)

24. A method of determining the presence of nephrotic syndrome in a patient, comprising:
a. obtaining a urine sample from the patient,
b. incubating the urine sample in a reaction mixture comprising:
i. a thermostable DNA polymerase having strand displacement activity, no 5′-3′ exonuclease activity, and which elongates from an RNA primer;
ii. deoxyribonucleotide triphosphates;
iii. a nucleic acid or nucleic acid analog probe comprising a 3′ portion and a 5′ portion, wherein the 3′ portion is a sequence that binds to or is fully complementary to a sequence 5′-UGUAAACAUCCUCGACUGGAAG-3′ (SEQ ID NO: 3), and the 5′ portion comprises in a 3′ to 5′ direction a first primer binding site, a second primer binding site spaced apart from the first binding site, and a third primer binding site;
iv. a first primer having a 3′ portion and a 5′ portion and a first tag at its 5′ end, where the 3′ portion of the first primer binds to or is fully complementary to the first primer binding site and the 5′ portion does not bind to the probe;
v. a second primer having a 3′ portion and a 5′ portion and a second tag, the same as or different from the first tag, at its 5′ end, where the 3′ portion of the second primer has the sequence of the second binding site or binds to a sequence fully complementary to the second binding site and the 5′ portion does not bind to a sequence fully complementary to the probe; and
vi. a third primer either having the sequence of the third binding site or binding a sequence complementary to the third binding site, and a third tag at its 5′ end that is different from the first and second tags;
c. incubating the reaction mixture at a temperature effective to produce reaction product from the polymerase in the presence of the target nucleic acid; and
d. determining the presence of, and optionally quantifying production of reaction product in the sample by detecting and/or quantifying nucleic acids larger than the first and second primer tagged with the first and second tags.

25. The method of claim 24, in which,
the probe has the sequence:
(SEQ ID NO: 1)
5′-GTTTCCCGTT CTAACGGAGT TTTGGAGGGC GAACGCGGCT
TTCCACTAGC TAGGCCCTAC ATGTCTTTCT TCCAGTCGAG
GATGTTTACA-3′;
the first primer has the sequence:
(SEQ ID NO: 2)
5′ CCACTAGCTATGTGACATGTAGGG-3′;
the second primer has the sequence:
(SEQ ID NO: 7)
5′ GCCGCGTTCTGTAGGAGGGC 3′;
and/or
the third primer has the sequence:
(SEQ ID NO: 5)
5′ GTTTCCCGTTCTAACGGAGT 3′.

26. The method of claim 24, in which,
the probe has the sequence:
(SEQ ID NO: 1)
5′-GTTTCCCGTT CTAACGGAGT TTTGGAGGGC GAACGCGGCT
TTCCACTAGC TAGGCCCTAC ATGTCTTTCT TCCAGTCGAG
GATGTTTACA-3′;
the first primer has the sequence:
(SEQ ID NO: 2)
5′ CCACTAGCTATGTGACATGTAGGG-3′;
the second primer has the sequence:
(SEQ ID NO: 7)
5′ GCCGCGTTCTGTAGGAGGGC 3′;
and
the third primer has the sequence:
(SEQ ID NO: 5)
5′ GTTTCCCGTTCTAACGGAGT 3′.
